(19) United States
(12) Patent Application Publication
(10) Pub. No.: US 2008/0183235 A1
(43) Pub. Date: Jul. 31, 2008

(54) INSULATIVE SHROUD FOR PLATE-TYPE ELECTRODES ADAPTED FOR CHRONIC IMPLANTATION

(76) Inventors: Christopher C. Stancer, Prescott, WI (US); Michael D. Fletcher, Minneapolis, MN (US)

Correspondence Address: MEDTRONIC, INC., 710 MEDTRONIC PARKWAY NE, MINNEAPOLIS, MN, 55432-9924, US

(21) Appl. No.: 11/669,519

(22) Filed: Jan. 31, 2007

(51) Int. Cl. A61N 1/375

(52) U.S. Cl. 607/36

(57) ABSTRACT

The invention provides diverse ways to reduce if not eliminate the possibility of an errant electrical path from one or more conductors of the shroud to a portion of the conductive housing of its corresponding IMD. That is—as described hereinbelow prior to backfilling the shroud with medical adhesive as a final fabrication step—according to various forms of the invention biocompatible electrical insulation is added between an IMD housing and the elongated conductors that couple to the surface electrodes. For example, a parylene coating can be added to the outside of the IMD housing using a simple spraying procedure and/or dip coating the IMD housing into the parylene. Parylene is a proven biocompatible, electrically insulative material. In addition to or in lieu of a coating or layer of parylene, a dielectric layer such as titanium nitride (TiN) or the like can be sputtered to the elongated conductors. Similarly, a layer of other materials could be added to the housing of the IMD and/or to the elongated conductors.

### CROSS REFERENCE TO RELATED APPLICATIONS

The present patent document is related to co-pending non-provisional patent applications; namely, Ser. No. 11/085,843, entitled, “APPARATUS AND METHODS OF MONITORING CARDIAC ACTIVITY UTILIZING IMPLANTABLE SHROUD-BASED ELECTRODES,” filed on 22 Mar. 2005 and Ser. No. 11/380,811 entitled, “SHROUD-BASED ELECTRODES HAVING VENTED GAPS,” filed 28 Apr. 2006, the contents of which are hereby fully incorporated by reference herein.

### FIELD OF THE INVENTION

The present invention relates generally to implantable medical devices (IMDs) and more particularly to a subcutaneous multiple electrode sensing and recording system for acquiring electrocardiographic data and waveform tracings from an implanted medical device without the need for or use of surface (skin) electrodes. More particularly, the present invention relates to implantable devices that are equipped with a shroud member fabricated in a manner to reduce potential for electrical shorting to a conductive housing of an IMD.

### BACKGROUND OF THE INVENTION

The electrocardiogram (ECG) is commonly used in medicine to determine the status of the electrical conduction system of the human heart. As practiced the ECG recording device is commonly attached to the patient via ECG leads connected to pads arrayed on the patient's body so as to achieve a recording that displays the cardiac waveforms in any one of 12 possible vectors.

Since the implantation of the first cardiac pacemaker, implantable medical device technology has advanced with the development of sophisticated, programmable cardiac pacemakers, pacemaker-cardioverter-defibrillator arrhythmia control devices and drug administration devices designed to detect arrhythmias and apply appropriate therapies. The detection and discrimination between various arrhythmic episodes in order to trigger the delivery of an appropriate therapy is of considerable interest. Prescription for implantation and programming of the implanted device are based on the analysis of the PQRST electrocardiogram (ECG) that currently requires externally attached electrodes and the electrogram (EGM) that requires implanted pacing leads. The waveforms are usually separated for such analysis into the P-wave and R-wave in systems that are designed to detect the depolarization of the atrium and ventricle respectively. Such systems employ detection of the occurrence of the P-wave and R-wave, analysis of the rate, regularity, and onset of variations in the rate of recurrence of the P-wave and R-wave, the morphology of the P-wave and R-wave and the direction of propagation of the depolarization represented by the P-wave and R-wave in the heart. The detection, analysis and storage of such EGM data within implanted medical devices are well known in the art. For example, S-T segment changes can be used to detect an ischemic episode. Acquisition and use of ECG tracing(s), on the other hand, has generally been limited to the use of an external ECG recording machine attached to the patient via surface electrodes of one sort or another.

The aforementioned ECG systems that utilize detection and analysis of the PQRST complex are all dependent upon the spatial orientation and number of electrodes available in or around the heart to pick up the depolarization wave front

As the functional sophistication and complexity of implantable medical device systems increased over the years, it has become increasingly more important for such systems to include a system for facilitating communication between one implanted device and another implanted device and/or an external device, for example, a programming console, monitoring system, or the like. For diagnostic purposes, it is desirable that the implanted device be able to communicate information regarding the device's operational status and the patient's condition to the physician or clinician. State of the art implantable devices are available which can even transmit a digitized electrical signal to display electrical cardiac activity (e.g., an ECG, EGM, or the like) for storage and/or analysis by an external device. The surface ECG, in fact, has remained the standard diagnostic tool since the very beginning of pacing and remains so today.

To diagnose and measure cardiac events, the cardiologist has several tools from which to choose. Such tools include twelve-lead electrocardiograms, exercise stress electrocardiograms, Holter monitoring, radioisotope imaging, coronary angiography, myocardial biopsy, and blood serum enzyme tests. Of these, the twelve-lead electrocardiogram (ECG) is generally the first procedure used to determine cardiac status prior to implanting a pacing system; thereafter, the physician will normally use an ECG available through the programmer to check the pacemaker's efficacy after implantation. Such ECG tracings are placed into the patient's records and used for comparison to more recent tracings. It must be noted, however, that whenever an ECG recording is required (whether through a direct connection to an ECG recording device or to a pacemaker programmer), external electrodes and leads must be used.

Unfortunately, surface electrodes have some serious drawbacks. For example, electrocardiogram analysis performed using existing external or body surface ECG systems can be limited by mechanical problems and poor signal quality. Electrodes attached externally to the body are a major source of signal quality problems and analysis errors because of susceptibility to interference such as muscle noise, power line interference, high frequency communication equipment interference, and baseline shift from respiration or motion. Signal degradation also occurs due to contact problems, ECG waveform artifacts, and patient discomfort. Externally attached electrodes are subject to motion artifacts from positional changes and the relative displacement between the skin and the electrodes. Furthermore, external electrodes require special skin preparation to ensure adequate electrical contact. Such preparation, along with positioning the electrode and attachment of the ECG lead to the electrode needlessly prolongs the pacemaker follow-up session. One possible approach is to equip the implanted pacemaker with the ability to detect cardiac signals and transform them into a tracing that is the same as or comparable to tracings obtainable via ECG leads attached to surface electrodes.

Previous art describes how to monitor electrical activity of the human heart for diagnostic and related medical purposes. U.S. Pat. No. 4,023,565 issued to Ohlsson describes circuitry for recording ECG signals from multiple lead inputs. Similarly, U.S. Pat. No. 4,263,919 issued to Levin, U.S. Pat. No. 4,170,227 issued to Feldman, et al, and U.S. Pat. No. 4,593,702 issued to Kepski, et al, describe multiple electrode systems, which combine surface EKG signals for artifact rejection.

The primary use for multiple electrode systems in the prior art is vector cardiography from ECG signals taken from multiple chest and limb electrodes. This is a technique whereby the direction of depolarization of the heart is monitored, as well as the amplitude. U.S. Pat. No. 4,121,576 issued to Greensite discusses such a system.

Numerous body surface ECG monitoring electrode systems have been employed in the past in detecting the ECG and conducting vector cardiographic studies. For example, U.S. Pat. No. 4,082,086 to Page, et al., discloses a four electrode orthogonal array that may be applied to the patient's skin both for convenience and to ensure the precise orientation of one electrode to the other. U.S. Pat. No. 3,983,867 to Case describes a vector cardiography system employing ECG electrodes disposed on the patient in normal locations and a hex axial reference system orthogonal display for displaying ECG signals of voltage versus time generated across sampled bipolar electrode pairs.

With regard to various aspects of time-release of surface coatings and the like for chronically implanted medical devices, the following issued patents are incorporated herein by reference. U.S. Pat. No. 6,997,949 issued 14 Feb. 2006 and entitled, “Medical device for delivering a therapeutic agent and method of preparation,” and U.S. Pat. No. 4,506,680 entitled, “Drug dispensing body implantable lead.” In the former patent, the following is described (from the Abstract section of the '949 patent) as follows: A device useful for localized delivery of a therapeutic agent is provided. The device includes a structure including a porous polymeric material and an elutable therapeutic agent in the form of a solid, gel, or neat liquid, which is dispersed in at least a portion of the porous polymeric material. Methods for making a medical device having blood-contacting surface electrodes is also provided.

Moreover, in regard to subcutaneously implanted EGM electrodes, the aforementioned Lindemans U.S. Pat. No. 4,310,000 discloses one or more reference sensing electrode positioned on the surface of the pacemaker case as described above. U.S. Pat. No. 4,313,443 issued to Lund describes a subcutaneously implanted electrode or electrodes for use in monitoring the ECG. Finally, U.S. Pat. No. 5,331,966 to Bennett, incorporated herein by reference, discloses a method and apparatus for providing an enhanced capability of detecting and gathering electrical cardiac signals via an array of relatively closely spaced subcutaneous electrodes (located on the body of an implanted device).

### SUMMARY

The present invention provides a leadless subcutaneous (or submuscular) single or multiple-electrode array that provides various embodiments of a compliant surround shroud coupled to a peripheral portion of an implantable medical device (IMD). The shroud incorporates a plurality of substantially planar electrodes mechanically coupled within recessed portions of the shroud. These electrodes electrically couple to circuitry of an IMD and are adapted to detect cardiac activity of a subject. Temporal recordings of the detected cardiac activity are referred to herein as an extra-cardiac electrogram (EC-EGM). The recordings can be stored upon computer readable media within an IMD at various resolution (e.g., continuous beat-by-beat, periodic, triggered, mean value, average value, etc.). Real time or stored EC-EGM signals can be provided to remote equipment via telemetry. For example, when telemetry, or programming, head of an IMD programming apparatus is positioned within range of an IMD the programmer receives some or all of the EC-EGM signals.

The invention provides diverse ways to reduce if not eliminate the possibility of an errant electrical path from one or more conductors of the shroud to a portion of the conductive housing of its corresponding IMD. That is—as described hereinbelow prior to backfilling the shroud with medical adhesive as a final fabrication step—according to various forms of the invention biocompatible electrical insulation is added between an IMD housing and the elongated conductors that couple to the surface electrodes. For example, a parylene coating can be added to the outside of the IMD housing using a simple spraying procedure and/or dip coating the IMD housing into the parylene. Parylene is a proven biocompatible, electrically insulative material. In addition to or in lieu of a coating or layer of parylene, a dielectric such as titanium nitride (TiN) can be sputtered to the elongated conductors. Similarly, a layer of other materials could be added to the housing of the IMD and/or to the elongated conductors. Examples include titanium oxide (TiO2) and zirconium oxide (ZrO2). Furthermore, a layer of urethane (solvent plus a medical adhesive such as Tecothane®) or silicone (another solvent-thinned medical adhesive) can be applied directly to the outside of the IMD housing, directly on the unassembled elongated conductors, and/or directly to the inside of a shroud assembly (with the plate electrodes already assembled). Another aspect of the invention implements further electrical insulation via electrical shrink tubing mechanically coupled to the elongated conductors (applied prior to assembly into the shroud assembly). The shrink tubing would of course need to also be biocompatible (e.g., a ptfe material could be used). In a related aspect a layer of biocompatible insulative self-adhesive tape can be coupled to the perimeter of the IMD housing (prior to attaching the shroud assembly to the IMD). In this form of the invention the self-adhesive tape would be completely covered with medical-grade adhesive and/or the body of the shroud assembly itself.

The diverse materials contemplated by the instant invention can be applied using any of a variety of techniques such as by sputtering, spraying, thermal or piezoelectric ink jet printing, electron beam deposition, immersion techniques, CVD or the like.

The present invention provides improved apparatus and methods for reliably collecting EC-EGM signals for use or collection in conjunction with diverse IMDs (e.g., implantable pacemakers having endocardial leads, implantable cardioverter-defibrillators or ICDs, drug delivery pumps, subcutaneous ICDs, submuscular ICDs, brain stimulation devices, nerve stimulation devices, muscle stimulation devices and the like).

The invention can be implemented employing suitable sensing amplifiers, switching circuits, signal processors, and memory to process the EC-EGM signals collected between any selected pair or pairs of the electrodes deployed in an array around the periphery or surface of a housing of an IMD to provide a leadless, orientation-insensitive means for receiving the EC-EGM signals from the heart.

The shroud can comprise a non-conductive, bio-compatible material such as any appropriate resin-based material, urethane polymer, silicone, or relatively soft urethane that retains its mechanical integrity during manufacturing and prolonged exposure to body fluids. The shroud placed around the peripheral portions of an IMD can utilize a number of configurations (e.g., two, three, four recesses) for individual electrodes. However, a three-electrode embodiment appears to provide an improved signal-to-noise ratio than the dual- or four-electrode embodiment. And, embodiments having a single electrode pair appear much more sensitive (i.e., negatively) to orientation of the device relative to the heart than embodiments having more than a single pair of electrodes. Of course, embodiments of the invention using more than three electrodes increase complexity without providing a significant improvement in signal quality. In the three electrode embodiments, one form includes equal spacing between the electrodes (i.e., configured as an equilateral triangle).

Embodiments having electrodes connected to three sense-amplifiers that are hardwired to three electrodes can record simultaneous EC-EGM signals. Alternative embodiments employ electrodes on the face of the lead connector, or header module, and/or major planar face(s) of the pacemaker that may be selectively or sequentially coupled in one or more pairs to the terminals of one or more sense amplifiers to pick up, amplify and process the EC-EGM signals across each electrode pair. In one aspect, the EC-EGM signals from a first electrode pair are stored and compared to other electrode pair(s) in order to determine the optimal sensing vector. Following such an optimization procedure, the system can be programmed to chronically employ the selected subcutaneous EC-EGM signal vector.

With respect to the elongated conductor coupling the planar electrodes to operative circuitry within an IMD, the assembly includes a unitary member stamped from a plate of conductive material such as titanium. In one embodiment the unitary member comprises a pre-shaped partially serpentine workpiece having a slightly curvilinear (i.e., substantially planar) major plate portion, a transition portion, and a partially serpentine portion adapted to cooperate with the configuration of the pre-configured conductor pathway.

Electrode assemblies according to the invention can be used for chronic or acute extra-cardiac electrogram (EC-EGM) signal sensing collection and attendant heart rate monitoring, capture detection, arrhythmia detection, and the like as well as detection of myriad other cardiac insults (e.g., ischemia monitoring using S-T segment changes, pulmonary edema monitoring based upon impedance changes).

In addition, the surface of the electrode can be treated with one or more electrode coatings to enhance signal-conducting, de- and re-polarization sensing properties, and to reduce polarization voltages (e.g., platinum black, titanium nitride, titanium oxide, iridium oxide, carbon, etc.). That is the surface area of the electrode surfaces may be increased by techniques known in the art and/or can be coated with such materials as just described and equivalents thereof. All of these materials are known to increase the true electrical surface area to improve the efficiency of electrical performance by reducing wasteful electrode polarization, among other advantages.

Many of the embodiments of the inventive electrodes herein can provide a continuous electrical path free of welds or bonds on a portion of the planar electrode, the transition portion, the elongated conductor or the distal tip portion. Moreover, the electrode assembly according to the invention anchors to a shroud member free of any chemical or adhesive bonding materials that can cause excursions due to electro-active specie release to the electrode surface or portions thereof.

These and other advantageous aspects of the invention will be appreciated by those of skill in the art after studying the invention herein described, depicted and claimed. In addition, persons of skill in the art will appreciate insubstantial modifications of the invention that are intended to be expressly covered by the appended claims.

### BRIEF DESCRIPTION OF THE DRAWINGS

FIG. 1 is an elevational side view depicting an exemplary shroud assembly coupled to an IMD which illustrates electrical conductors disposed in the header, or connector, portion of the IMD which is configured to receive a proximal end portion of medical electrical leads (not shown).

FIG. 2 is a perspective view of the IMD depicted in FIG. 1 further illustrating the shroud assembly.

FIG. 3 is a perspective view of an opposing major side of the IMD depicted in FIGS. 1 and 2.

FIG. 4 is a plan view of the IMD previously depicted that illustrates the relationship between two of the electrodes coupled to the shroud assembly as well as depicting the header, or connector, of the IMD.

FIG. 5 is a photocopy copy of a first side of a transparent shroud assembly coupled to a header according to the invention that clearly illustrates that the conductors and components of the assembly are readily visible.

FIG. 6 is a photocopy copy of a second side of the transparent shroud assembly coupled to a header according to the invention that clearly illustrates that the conductors and components of the assembly are readily visible from both sides.

### DETAILED DESCRIPTION OF THE DRAWINGS

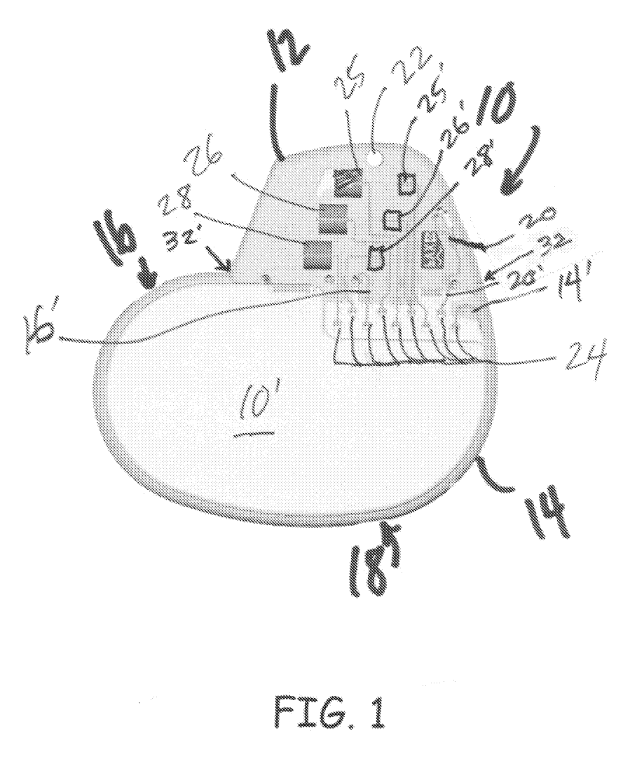

FIG. 1 is an elevational side view depicting an exemplary shroud assembly 14 coupled to an IMD 10 which illustrates electrical conductors 24,25,26,28 disposed in the header, or connector, portion 12 of the IMD 10 which are configured to couple to end portions of medical electrical leads as well as couple to operative circuitry within the IMD housing (not shown). The shroud assembly 14 surrounds IMD 10 and mechanically couples to the header portion 12 and includes at least three discrete electrodes 16,18,20 adapted for sensing far-field, or extra-cardiac electrogram (EC-EGM) signals. FIG. 1 also depicts an aperture 22 formed within the header 12 which can be used to receive thread used to suture the header 12 (and thus the IMD 10) to a fixed surgical location (also known as a pocket) of a patient's body.

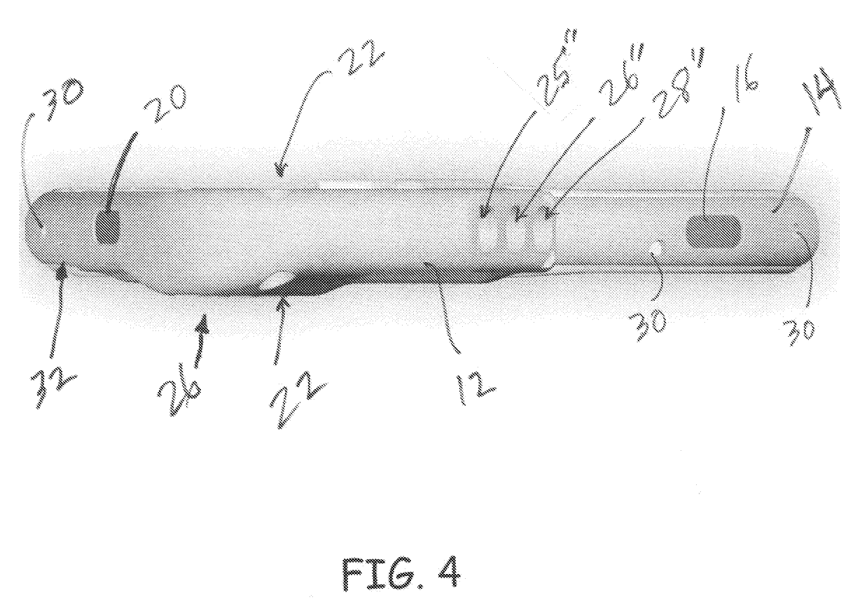

As partially depicted in FIG. 1, an elongated conductor 14′ couples to electrode 14, elongated conductor 16′ couples to electrode 16, and conductor segment 20′ couples to electrode 20. Furthermore, three of the conductors (denoted collectively with reference numeral 24) couple to three cuff-type conductors 25,26,28 adapted to receive proximal portions of medical electrical leads while another three of the conductors couple to conductive pads 25′,26′,28′ which are aligned with, but spaced from the conductors 25,26,28 along a trio of bores (denoted as 25″,26″,28″ in FIG. 4 herein) formed in header 12.

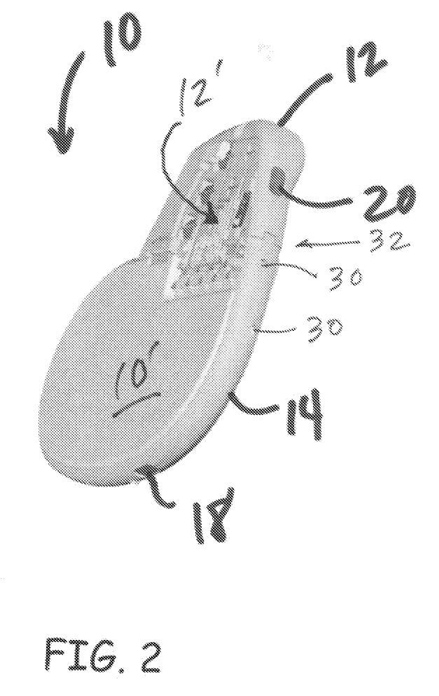

FIG. 2 is a perspective view of the IMD 10 depicted in FIG. 1 further illustrating the shroud assembly 14 and two of the three electrodes 18,20. In addition, two of a plurality of adhesive ports 30 and a mechanical joint 32 between the elongated portion of the shroud assembly 14 and the header 12 are also depicted in FIG. 2. The ports 30 can be used to evacuate excess medical adhesive disposed between the shroud assembly 14 and the IMD 10 and/or used to inject medical adhesive into one or more ports 30 to fill the void(s) therebetween. In one form of the invention, a major lateral portion 12′ of header 12 remains open to ambient conditions during assembly of the IMD 10. Subsequent to making electrical connections between the plurality of conductors of the shroud assembly 14 and the header 12, the open lateral portion 12′ is sealed (e.g., automatically or manually filled with a biocompatible substance such as a substantially clear medical adhesive, such as Tecothane® made by Noveon, Inc. a wholly owned subsidiary of The Lubrizol Corporation). Thus most if not all of the plurality of conductors of the shroud assembly 14 and the IMD 10 are visible and can be manually and/or automatically inspected to ensure long term operability and highest quality of the completed IMD 10.

Some properties of various Tecothane® appear below (as published in the Technical Data Sheet (TDS) for certain clear grades of the material:

Tecothane ® Typical Physical Test Data - CLEAR GRADES

| | ASTM Test | TT-1074A | TT-1085A | TT-1096A | TT-1055D | TT-1066D | TT-1069D | TT-1072D | TT-1075D-M |
|---|---|---|---|---|---|---|---|---|---|
| Durometer | D2240 | 75A | 85A | 94A | 54D | 64D | 69D | 74D | 75D |
| (Shore Hardness) | | | | | | | | | |
| Specific Gravity | D792 | 1.10 | 1.12 | 1.15 | 1.16 | 1.18 | 1.18 | 1.18 | 1.19 |
| Flexural Modulus | D790 | 1.300 | 3.000 | 8.000 | 18.000 | 26.000 | 44.000 | 73.000 | 190.000 |
| (psi) | | | | | | | | | |
| Ultimate Tensile | D412 | 6.000 | 7.000 | 9.000 | 9.600 | 10.000 | 8.800 | 9.000 | 8.300 |
| (psi) | | | | | | | | | |
| Ultimate Elongation | D412 | 550 | 450 | 400 | 350 | 300 | 310 | 275 | 150 |
| (%) | | | | | | | | | |
| Tensile (psi) | D412 | | | | | | | | |
| at 100% Elongation | | 500 | 800 | 1.300 | 2.500 | 2.860 | 3.200 | 3.700 | 3.600 |
| at 200% Elongation | | 700 | 1.000 | 2.100 | 3.800 | 4.600 | 4.200 | 3.900 | NA |
| at 300% Elongation | | 1.100 | 1.600 | 4.300 | 6.500 | 7.800 | NA | NA | NA |
| Melt Index | D1238 | 3.5 | 4.0 | 3.8 | 4.0 | 2.0 | 3.0 | 2.0 | 5.0 |
| (gm 10 min at | | (205° C.) | (205° C.) | (210° C.) | (210° C.) | (210° C.) | (210° C.) | (210° C.) | (210° C.) |
| 2160 gm load) | | | | | | | | | |
| Mold Shrinkage (in/in) | D.955 | .008–.012 | .009–.012 | .006–.010 | .004–.006 | .004–.008 | .004–.008 | .004–.006 | .004–.006 |

Referring again to FIG. 2, the terminal ends of conductors 24 are depicted to include the optional shaped-end portion which provides a target for reliable automatic and/or manual coupling (e.g., laser welding, soldering, and the like) of the terminal end portions to respective conductive pins of a multi-polar feedthrough assembly (not shown). As is known in the art, such conductive pins hermetically couple to operative circuitry disposed within the IMD 10.

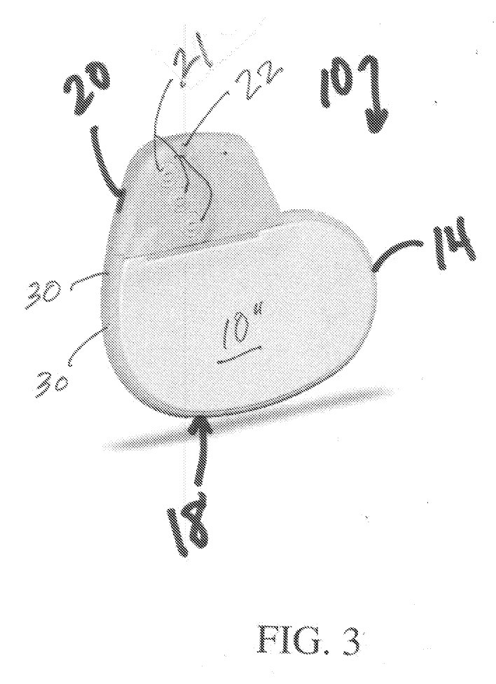

FIG. 3 is a perspective view of an opposing major side 10″ of the IMD 10 depicted in FIGS. 1 and 2 and three self-healing grommets 21 substantially hermetically coupled to openings of a like number of threaded bores (shown in FIG. 6 and denoted by reference numeral 26′). As is known, the threaded bores are configured to receive a threaded shank and the grommets 21 are fabricated to temporarily admit a mechanical tool (not shown). The tool is used to connect and allow a physician or clinician to manually tighten the conductors 25,26,28, for example, with compression and/or radially around conductive rings disposed on proximal portions of medical electrical leads (not shown). In addition, two of the plurality of ports 30 are also depicted in FIG. 3.

FIG. 4 is a plan view of the IMD 10 previously depicted that illustrates the relationship between two of the electrodes 16,20 coupled to the shroud assembly 14 as well as depicting the header 12, or connector, of the IMD 10. Opposing openings of the aperture 22 formed in the header 12 are also depicted in FIG. 4 as are the three openings 25″,26″,28″ of the bores or ports formed in the header 12 that are configured to admit the proximal end of medical electrical leads (not shown). Three of the adhesive-admitting ports 30 are shown distributed at various locations through the surfaces of the shroud 14.

Three elongated conductors individually couple to a respective electrode 14,16,18. These elongated conductors can be continuous or discrete segments of conductive material. In the event that they comprise discrete segments, they need to be coupled together such as with convention means like laser bonding, welding, soldering and the like. For example, the elongated conductor coupling to electrode 16 can traverse either direction around the periphery of the IMD 10 disposed within or mechanically coupled to an inner portion of the shroud 14. If it traverses past the seam 32 it might need to be isolated from the elongated conductor coupled to electrode 18 (assuming that conductor also traversed seam 32). If the conductor coupling electrode 16 is routed directly toward the header 12 (and the header/shroud is not a unitary structure) then a bond between segments of the elongated conductor could be necessary at the junction of the shroud 14 and the header 12.

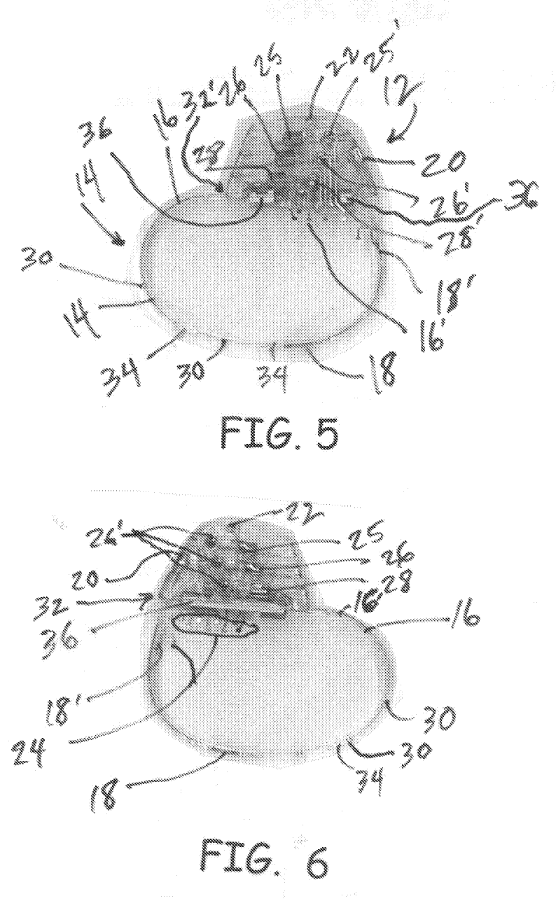

FIG. 5 is a photocopy copy of a first side of a transparent shroud assembly 14 coupled to a header 12 according to the invention that clearly illustrates that the conductors and components of the assembly are readily visible. FIG. 6 is a photocopy copy of a second side of the transparent shroud assembly coupled to a header according to the invention that clearly illustrates that the conductors and components of the assembly are readily visible from both sides.

Since FIG. 5 and FIG. 6 essentially depict common components of the inventive assembly of the invention they shall be described together. The exemplary shroud assembly 14 of FIGS. 5 and 6 is depicted with an IMD 10 for clarity. The electrical conductors 25,26,28 disposed in the header, or connector, portion 12 of the IMD 10 are configured to couple to end portions of medical electrical leads as well as couple to operative circuitry within the IMD housing (not shown). The shroud assembly 14 mechanically couples to the header portion 12 at each end of the shroud assembly 14 both mechanically and electrically via medical adhesive (disposed at overlapping joint 32′) and an elongate conductor 16′ (passing through joint 32′). The three discrete electrodes 16,18,20 and their corresponding elongated conductors 16′,18′, 20′ are coupled together. While not depicted in FIGS. 5 and 6 the conductors 16′,18′,20′ have at least a partially serpentine configuration and conductors 16′,18′ are furthermore mechanically coupled to the shroud with a series of elongated stand-off bosses 34. In addition, and as previously mentioned, during attachment to an IMD adhesive is disposed intermediate the shroud 14 and the IMD with excess being evacuated from ports 30 (and/or if needed injected into one of more ports 30) to eliminate any air bubbles. Of course, one feature of the invention relates to the ability to fully inspect the finished article visually (including the quality of the electrical connections and the quality of the bond between the shroud 14 and an IMD. Also, the electrodes 16,18 can be at least one of mechanically embedded partially into the material of the shroud 14 and configured to receive medical adhesive to retain the electrodes in position (e.g., using perforated wing-like peripheral portions of the electrodes disposed at the ends, sides, and/or other parts of the periphery of an electrode). Aperture 22 also can be seen in FIGS. 5 and 6 formed in a peripheral portion of the header 12. Also depicted is how the elongated conductor 14′ couples to electrode 14, elongated conductor 16′ couples to electrode 16, and conductor segment 20′ couples to electrode 20. Furthermore, three of the conductors (denoted collectively with reference numeral 24) couple to three cuff-type conductors 25,26,28 adapted to receive proximal portions of medical electrical leads while another three of the conductors couple to conductive pads 25′,26′,28′ which are aligned with, but spaced from the conductors 25,26,28 along a trio of bores (denoted as 25″,26″,28″ in FIG. 4 herein) formed in header 12. The joint 32 between header 12 and shroud 14 can comprise a variety of mechanisms, including an interlocking, partially spring-biased socket-type connection which, in combination with medical adhesive, provides a reliable mechanical coupling.

Another feature of the invention relates to including radio-opaque markers and/or identifiers within and/or on the shroud 14 so that a physician or clinician can readily determine that an IMD is outfitted with an assembly according to this invention. A marker according to this aspect of the invention can include a metallic insert and/or coating having a unique shape, location and/or configuration (e.g., an “M” or the corporate logo for an IMD manufactured by Medtronic, Inc.).

Depicted in FIGS. 5 and 6 is an elongated structural support member 36 which provides a reliable connection to a metallic housing of an IMD (not shown) via traditional processes (e.g., laser welding). The member 36 has a three substantially orthogonal sides (all denoted as 36 in FIGS. 5 and 6) thus providing three discrete bonding areas between the header 12 and an IMD. Of course, the member 36 could be perforated and/or coated with an insulative material, but in the embodiment depicted one side is cut out or not present so that the plurality of conductors 24 can pass from the header 12 and shroud 14 to the feedthrough array of the IMD.

Electrodes 16,18,20 and/or the (corresponding elongated conductors) can be fabricated out of any appropriate material, including without limitation tantalum, tantalum alloy, titanium, titanium alloy, platinum, platinum alloy, or any of the tantalum, titanium or platinum group of metals whose surface may be treated by sputtering, platinization, ion milling, sintering, etching, or a combination of these processes to create a large specific surface area. Also as noted herein, an electrode can be stamped, drawn, laser cut or machined using electronic discharge apparatus. Some of the foregoing might require de-burring of the periphery of the electrode or alternately any sharp edges due to a burr can be coupled facing toward the corresponding recess in the shroud member thereby minimizing likelihood of any patient discomfort post-implant while further reducing complexity in the fabrication of assemblies according to the invention. The electrodes can be coated or covered with platinum, a platinum-iridium alloy (e.g., 90:10), platinum black, titanium nitride or the like.

Accordingly, a number of embodiments and aspects of the invention have been described and depicted although the inventors consider the foregoing as illustrative and not limiting as to the full reach of the invention. That is, the inventors hereby claim all the expressly disclosed and described aspects of the invention as well as those slight variations and insubstantial changes as will occur to those of skill in the art to which the invention is directed. The following claims define the core of the invention and the inventors consider said claims and all equivalents of said claims and limitations thereof to reside squarely within their invention.

1. A subcutaneous cardiac activity sensing device, comprising:
an implantable medical device (IMD), wherein the exterior surface of the IMD housing is rendered electrically insulative;
a shroud member subsequently coupled to the part of the periphery of the IMD;
a header portion subsequently coupled to the shroud member and to a different portion of the periphery of the IMD;
at least one electrically insulated elongated conductor coupled to a hermetic feedthrough pin disposed between the IMD housing and the header portion; and
at least one electrode mechanically coupled to the elongated conductor and the shroud member.

2. A device according to claim 1, wherein said shroud member comprises a resilient material.

3. A device according to claim 1, wherein the outside of the IMD housing is coated with an electrically insulative parylene material.

4. A device according to claim 3, the parylene material is applied to the IMD housing via one of dip coating and spraying.

5. A device according to claim 1, wherein the at least one electrically insulated elongated conductor is coated with one of a layer of a titanium oxide material and a layer of a zirconium oxide material.

6. A device according to claim 5, wherein the shroud member comprises a coat of one of an electrically insulative solvent-thinned urethane material and an electrically insulative solvent-thinned silicone material.

7. A device according to claim 6, further comprising a volume of substantially clear medical adhesive disposed between the interior surface portion of the shroud member and the periphery of the IMD.

8. A device according to claim 7, further comprising a segment of electrically insulative tubing that shrinks upon application of thermal stress surrounding at least a portion of the elongated conductor.

9. A device according to claim 1, wherein the outside perimeter of the IMD housing comprises a layer of self-adhesive insulative tape disposed intermediate the surface of the housing and the elongated conductor.

10. A device according to claim 9, wherein the at least one electrode comprises three electrodes each disposed in spaced apart relation and each coupled to the shroud member.

11. A device according to claim 10, wherein the three electrodes are disposed in an approximately equally spaced apart triangular relationship.

12. A device according to claim 1, further comprising a suture-receiving aperture formed through the header portion.

13. A device according to claim 3, wherein the elongated conductor comprises a structure integrally formed with the electrode.

14. A device according to claim 1, wherein the at least a pair of electrodes are fabricated from one of a titanium material and a platinum material.

15. A device according to claim 14, wherein the at least a pair of electrodes further includes a coating on at least a major surface thereof.

16. A device according to claim 15, wherein the coating comprises one of an electrically insulative solvent-thinned urethane material and an electrically insulative solvent-thinned silicone material.

17. A device according to claim 1, further comprising medical grade adhesive disposed between the shroud member and a part of the periphery of the IMD.

18. A device according to claim 1, wherein the IMD comprises one of: an implantable cardiac pacemaker, an implantable cardioverter-defibrillator, an implantable fluid delivery device, an implantable neurostimulator, an implantable gastric simulator.

19. A method of fabricating a cardiac sensing shroud assembly, comprising:
rendering a conductive housing for an implantable medical device (IMD) electrically insulative;
providing a resilient shroud member adapted to be mounted around at least a part of the peripheral portion of the housing;
forming an aperture in a peripheral portion of the shroud member; and
coupling a substantially flat surfaced electrode into engagement in the aperture of the resilient substantially clear shroud.

20. A method according to claim 18, further comprising: coupling the shroud member around at least a part of the peripheral portion of the IMD,
wherein the IMD comprises one of: an implantable cardiac pacemaker, an implantable cardioverter-defibrillator, an implantable fluid delivery device, an implantable neurostimulator, an implantable gastric simulator.
